(19) United States
(12) Patent Application Publication
(10) Pub. No.: US 2026/0228387 A1
(43) Pub. Date: Aug. 6, 2026

(54) ANTI-BUCKLING SYSTEM INTEGRATING STRESS-DRIVEN SELF-CENTERING AND CATHODIC PROTECTION FUNCTIONS AND DESIGN METHOD THEREOF

(71) Applicant: Shenzhen University, Shenzhen (CN)

(72) Inventors: Xiaoxu HUANG, Shenzhen (CN); Biao HU, Shenzhen (CN); Zhongfeng ZHU, Shenzhen (CN); Menghuan GUO, Shenzhen (CN)

(21) Appl. No.: 19/456,157

(22) Filed: Jan. 22, 2026

(30) Foreign Application Priority Data
Feb. 5, 2025 (CN) 202510128407.0

(51) Int. Cl. G06F 30/20; G06F 113/26; G06F 119/14

(52) U.S. Cl. CPC G06F 30/20; G06F 2113/26; G06F 2119/14

(57) ABSTRACT

Provided are an anti-buckling system integrating stress-driven self-centering and cathodic protection functions and a design method thereof. The anti-buckling system includes a composite tendon, a power source, and a sensing assembly. The composite tendon includes a plurality of diagonal tension assemblies. Each of the diagonal tension assemblies includes a shape memory alloy (SMA) structure and carbon fiber-reinforced polymer (CFRP) filaments that are insulated from each other. The CFRP filaments are disposed in a groove of the SMA core. Two ends of the SMA core in the diagonal tension assemblies are connected to a positive electrode and a negative electrode of the power source, respectively, and the CFRP filaments in the diagonal tension assemblies are connected to the positive electrode of the power source. The steel structure column is connected to the negative electrode of the power source. This application significantly enhances the durability and anti-buckling performance of steel structures.

### CROSS REFERENCE TO RELATED APPLICATION

This patent application claims the benefit and priority of Chinese Patent Application No. 2025101284070, filed with the China National Intellectual Property Administration on Feb. 5, 2025, the disclosure of which is incorporated by reference herein in its entirety as part of the present application.

### TECHNICAL FIELD

The present disclosure relates to the technical field of steel structure protection, and in particular, to an anti-buckling system integrating stress-driven self-centering and cathodic protection functions and a design method thereof.

### BACKGROUND

The buckling resistance of steel structures plays a crucial role in the safety and durability of buildings, especially when the steel structures bear lateral loads and seismic actions. Existing anti-buckling devices, such as traditional stiffening members, external support frames, and damping devices, have improved the stability of components to a certain extent, but still have some deficiencies. For example, traditional stiffening devices have poor corrosion resistance, are susceptible to damage, particularly in humid or corrosive environments, and have high maintenance costs; damping devices are often non-recoverable to their original state after an earthquake, have limited energy dissipation characteristics, and are difficult to adapt to high-intensity external impacts; furthermore, many devices have poor ductility and are prone to brittle failure under large deformations.

Therefore, the prior art has yet to be improved and developed.

### SUMMARY

A primary objective of the present disclosure is to provide an anti-buckling system integrating stress-driven self-centering and cathodic protection functions and a design method thereof, aiming to solve the problem that in the prior art, when steel structures achieve buckling resistance through anti-buckling devices in coastal environments and under seismic loads, the devices have poor corrosion resistance, impact resistance or ductility, resulting in insufficient corrosion resistance of the steel structures.

According to a first aspect, an embodiment of the present disclosure provides an anti-buckling system integrating stress-driven self-centering and cathodic protection functions. The anti-buckling system integrating stress-driven self-centering and cathodic protection functions includes a composite tendon, a power source, and a sensing assembly. The composite tendon and the sensing assembly are installed on a steel structure column. The composite tendon includes a plurality of diagonal tension assemblies, and the plurality of diagonal tension assemblies are symmetrically distributed on two sides of the steel structure column. Each of the diagonal tension assemblies includes a shape memory alloy (SMA) structure and a fiber-reinforced composite structure that are insulated from each other. The fiber-reinforced composite structure is disposed in a groove of the SMA structure. Two ends of the SMA structure corresponding to each of the diagonal tension assemblies are connected to a positive electrode and a negative electrode of the power source, respectively, and the fiber-reinforced composite structure corresponding to each of the diagonal tension assemblies is connected to the positive electrode of the power source. The steel structure column is connected to the negative electrode of the power source. When the sensing assembly detects that a strain of the steel structure column reaches a preset condition, the power source applies an excitation current to the SMA structure corresponding to the diagonal tension assembly under tension, such that the diagonal tension assembly under tension recovers to a target length.

Optionally, in an embodiment of the present disclosure, a first lateral support and a second lateral support are provided on two sides of the steel structure column, respectively. The plurality of diagonal tension assemblies includes a first diagonal tension member, a second diagonal tension member, a third diagonal tension member, and a fourth diagonal tension member. Two ends of the first diagonal tension member are connected to a top end of the steel structure column and an upper side end of the first lateral support respectively; two ends of the second diagonal tension member are connected to the top end of the steel structure column and an upper side end of the second lateral support respectively; two ends of the third diagonal tension member are connected to a bottom end of the steel structure column and a lower side end of the first lateral support respectively; two ends of the fourth diagonal tension member are connected to the bottom end of the steel structure column and a lower side end of the second lateral support respectively.

Optionally, in an embodiment of the present disclosure, the sensing assembly includes a first strain sensor and a second strain sensor. The first strain sensor is disposed on the first diagonal tension member, and the second strain sensor is disposed on the second diagonal tension member. When the first strain sensor detects that a strain of the first diagonal tension member is greater than a preset value, the power source applies an excitation current to the first diagonal tension member and the third diagonal tension member, such that the first diagonal tension member and the third diagonal tension member recover to an initial length. When the second strain sensor detects that a strain of the second diagonal tension member is greater than the preset value, the power source applies an excitation current to the second diagonal tension member and the fourth diagonal tension member, such that the second diagonal tension member and the fourth diagonal tension member recover to the initial length.

Optionally, in an embodiment of the present disclosure, the groove is filled with an insulating layer, and the fiber-reinforced composite structure is insulated from the SMA structure through the insulating layer.

Optionally, in an embodiment of the present disclosure, each of the diagonal tension assemblies includes a reinforced segment, two variable cross-section segments, and two installation segments. The two installation segments are located at two ends; the reinforced segment is located in a middle position; each of the variable cross-section segments connects one end of the reinforced segment and one of the installation segments; and a diameter of the reinforced segment is smaller than a diameter of each of the installation segments.

Optionally, in an embodiment of the present disclosure, the groove is a spiral groove, a cross-section of the spiral groove is circular or rectangular, the spiral groove is used for anchoring the fiber-reinforced composite structure, and the fiber-reinforced composite structure has an ultimate strain range of 1.5%-2%.

According to a second aspect, an embodiment of the present disclosure further provides an anti-buckling design method of the anti-buckling system integrating stress-driven self-centering and cathodic protection functions described above. The anti-buckling design method includes: determining a size and material properties of the SMA structure, and establishing a relational expression between the SMA structure and the corresponding fiber-reinforced composite structure; determining an angle of the SMA structure and a usage amount of the fiber-reinforced composite structure according to the size, the material properties, and the relational expression; and installing, on the steel structure column, a plurality of diagonal tension assemblies each formed by the SMA structure and the corresponding fiber-reinforced composite structure according to the angle and the usage amount, and if stiffness of the steel structure column reaches a critical stiffness, completing anti-buckling design of one of the diagonal tension assemblies.

Optionally, in an embodiment of the present disclosure, the material properties include an axial strain at a necking section of the SMA structure, a corresponding cross-sectional pressure at the necking section, and a hardening modulus; the determining the size and the material properties of the SMA structure includes: determining the size of the SMA structure based on parameters of the steel structure column; and determining the axial strain at the necking section of the SMA structure, the corresponding cross-sectional pressure at the necking section, and the hardening modulus based on a stress-strain curve of the SMA structure.

Optionally, in an embodiment of the present disclosure, the relational expression is as follows:

$${A}_{f} {E}_{f} \{ cos α + \frac{ρ ( {ε}_{y , r} ) - 5 {tan}^{2} {α ( \frac{{r}_{b}}{{r}_{p}} )}^{2} + 5 {tan}^{2} α ( \frac{{r}_{b}}{{r}_{p}} )}{κ ( {ε}_{y , r} ) γ ( {ε}_{y , r} )} \} \geq [ \frac{{A}_{s}}{1 + {ε}_{y , r}} ( \frac{1}{1 + {ε}_{y , r}} ) {σ}_{s} ( {ε}_{y , r} ) - {E}_{t} ] ;$$

where A_{f} is an area of the fiber-reinforced composite structure; E_{f} is an elastic modulus of the fiber-reinforced composite structure; α is a winding angle of the fiber-reinforced composite structure; ε_{y,r} is a strain at a necking section of the SMA structure; ρ(ε_{y,r}) is an axial strain conversion coefficient corresponding to the strain at the necking section of the SMA structure; r_{b} is a radius of the SMA structure; rₚ is a distance from a center of a cross-section of the SMA structure to a center of a cross-section of the fiber-reinforced composite structure; κ(ε_{y,r}) is an elastic modulus conversion coefficient of the fiber-reinforced composite structure corresponding to the strain at the necking section of the SMA structure; γ(ε_{y,r}) is an area conversion coefficient of the fiber-reinforced composite structure corresponding to the strain at the necking section of the SMA structure; As is a cross-sectional area of the SMA structure; σₛ(ε_{y,r}) is a stress corresponding to the strain at the necking section of the SMA structure; and Eₜ is the hardening modulus of the SMA structure.

Optionally, in an embodiment of the present disclosure, the critical stiffness is expressed as:

$${K}_{0} = [ σ ] {N}_{d} {l}^{2} / ( 12 {σ}_{d} )$$

where K₀ is the critical stiffness; [σ] is an allowable stress of the diagonal tension assembly; od is a design stress at two ends of the diagonal tension assembly; I is a height of a rectangular column; and N_{d} is a design axial pressure of the rectangular column.

Beneficial effects: The present disclosure provides an anti-buckling system integrating stress-driven self-centering and cathodic protection functions and a design method thereof. In the anti-buckling system, a diagonal tension assembly is formed by an SMA structure and a corresponding fiber-reinforced composite structure, and a plurality of diagonal tension assemblies are installed on a steel structure column. Strain is detected by a sensing assembly on the surface of the steel structure column, so that a power source applies an excitation current to the SMA structure in the diagonal tension assembly under tension to restore the diagonal tension assembly to an original target length. Thus, in coastal environments and under seismic loads, the steel structure column is protected by the various diagonal tension assemblies, improving the anti-corrosion performance of the structure, and the diagonal tension assemblies are capable of self-centering, improving the self-recovery capacity of the structure.

### BRIEF DESCRIPTION OF THE DRAWINGS

To describe the technical solutions in the embodiments of the present disclosure or in the prior art more clearly, the following briefly describes the accompanying drawings required for describing the examples or the prior art. Apparently, the accompanying drawings in the following description only show some embodiments of the present disclosure, and a person of ordinary skill in the art may still derive other drawings from these accompanying drawings without creative efforts.

FIG. 1 is a perspective view of a preferred embodiment of an anti-buckling system integrating stress-driven self-centering and cathodic protection functions according to the present disclosure;

FIG. 2 is a plan view of an SMA-carbon fiber reinforced polymer (CFRP) tendon in a preferred embodiment of an anti-buckling system integrating stress-driven self-centering and cathodic protection functions according to the present disclosure;

FIG. 3 is a schematic structural diagram of an SMA-CFRP tendon in a preferred embodiment of an anti-buckling system integrating stress-driven self-centering and cathodic protection functions according to the present disclosure;

FIG. 4 is a flowchart of a preferred embodiment of an anti-buckling design method according to the present disclosure;

FIG. 5 is a diagram illustrating a stress-strain relationship curve of SMA-CFRP in a preferred embodiment of an anti-buckling design method according to the present disclosure; and

FIG. 6 is a schematic diagram of a compression buckling resistance mechanism of an SMA-CFRP tendon in a preferred embodiment of an anti-buckling design method according to the present disclosure.

### REFERENCE NUMERALS

11. First diagonal tension member; 12. Second diagonal tension member; 13. Third diagonal tension member; 14. Fourth diagonal tension member.
20. Steel structure column; 21. First lateral support; 22. Second lateral support.

Specific embodiments of the present disclosure are shown by using the accompanying drawings and are described below in more detail. The accompanying drawings and text description are not intended to limit the scope of the concept of the present disclosure in any manner, but to explain the concept of the present disclosure for those skilled in the art with reference to specific embodiments.

### DETAILED DESCRIPTION OF THE EMBODIMENTS

To make the objectives, technical solutions, and effects of the present disclosure clearer, the following clearly and completely describes the technical solutions in embodiments of the present disclosure with reference to the accompanying drawings in the embodiments of the present disclosure. The described embodiments are merely some possible technical implementations rather than all possible implementations of the present disclosure. Based on the embodiments of the present disclosure, all other embodiments obtained by a person skilled in the art by combining the embodiments of the present disclosure without creative efforts shall fall within the protection scope of the present disclosure.

First, terms involved in the embodiments of the present disclosure are introduced: SMA is short for shape memory alloy, that is, a shape memory alloy structure in present disclosure is an SMA tendon; CFRP is short for carbon fiber reinforced polymer, that is, a fiber reinforced composite structure in present disclosure is a CFRP fiber; a diagonal tension assembly in present disclosure is an SMA-CFRP tendon, and a plurality of diagonal tension assemblies form a composite tendon.

The following describes the anti-buckling system integrating stress-driven self-centering and cathodic protection functions and a design method thereof according to the embodiments of the present disclosure with reference to the accompanying drawings. To address the problem that in the prior art, when steel structures achieve buckling resistance through anti-buckling devices in coastal environments and under seismic loads, the devices have poor corrosion resistance, impact resistance or ductility, resulting in insufficient corrosion resistance of the steel structures, the present disclosure provides an anti-buckling system integrating stress-driven self-centering and cathodic protection functions. In the anti-buckling system, a diagonal tension assembly is formed by an SMA structure and a corresponding fiber-reinforced composite structure, and a plurality of diagonal tension assemblies are installed on a steel structure column. Strain is detected by a sensing assembly on the surface of the steel structure column, so that a power source applies an excitation current to the SMA structure in the diagonal tension assembly under tension to restore the diagonal tension assembly to an original target length. Thus, in coastal environments and under seismic loads, the steel structure column is protected by the diagonal tension assemblies, improving the anti-corrosion performance of the structure, and the diagonal tension assemblies are capable of self-centering, improving the self-recovery capacity of the structure. Therefore, the present disclosure solves the technical problem that in the prior art, when steel structures achieve buckling resistance through anti-buckling devices in coastal environments and under seismic loads, the devices have poor corrosion resistance, impact resistance or ductility, resulting in insufficient corrosion resistance of the steel structures.

The anti-buckling system of the present disclosure, through the combination of a fiber-reinforced composite and shape memory alloy (SMA), achieves the advantages of high strength, high energy dissipation, and high ductility. Fiber-reinforced composites have the advantages of high strength and corrosion resistance, while the self-centering characteristic of SMA can help the device recover to its original state after deformation under force, thereby improving the self-recovery capacity of the structure. Furthermore, winding fiber-reinforced composites such as CFRP on the surface of the SMA can effectively inhibit the necking of the SMA and significantly improve the post-yield strength and large plastic deformation capacity of the SMA, which exhibits excellent buffering effects in resisting large-scale buckling failure of components. That is, the present disclosure can improve the anti-corrosion performance of the steel structure column in coastal environments and under seismic loads and its anti-buckling performance under external loads.

The technical solutions of the present disclosure will be described in detail below with reference to specific embodiments. The following specific embodiments may be combined with each other, and the same or similar concepts or processes may not be repeatedly described in some embodiments.

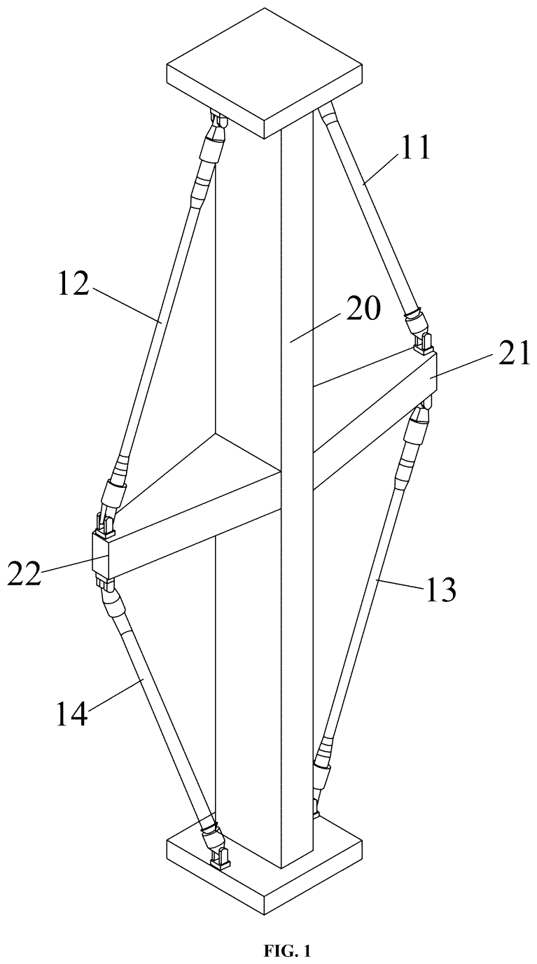

As shown in FIG. 1, an embodiment of the present disclosure provides an anti-buckling system integrating stress-driven self-centering and cathodic protection functions. The anti-buckling system integrating stress-driven self-centering and cathodic protection functions includes a composite tendon, a power source (not shown in the figure), and a sensing assembly (not shown in the figure). The composite tendon and the sensing assembly are installed on a steel structure column 20. The composite tendon includes a plurality of diagonal tension assemblies, and the plurality of diagonal tension assemblies are symmetrically distributed on two sides of the steel structure column 20. Each of the diagonal tension assemblies includes an SMA structure (that is, SMA core) and a fiber-reinforced composite structure (that is, fiber-reinforced composite filament) that are insulated from each other. The fiber-reinforced composite structure is disposed in a groove of the SMA structure. Two ends of the SMA structure corresponding to each of the diagonal tension assemblies are connected to a positive electrode and a negative electrode of the power source respectively, and the fiber-reinforced composite structure corresponding to each of the diagonal tension assemblies is connected to the positive electrode of the power source. The steel structure column 20 is connected to the negative electrode of the power source. When the sensing assembly detects that a strain of the steel structure column 20 reaches a preset condition, the power source applies an excitation current to the SMA structure corresponding to the diagonal tension assembly under tension, such that the diagonal tension assembly under tension recovers to a target length.

In the embodiment of the present disclosure, the plurality of diagonal tension assemblies (SMA-CFRP tendons) are divided into one part that is under tension and another part that is under compression. An excitation current is applied to the SMA tendon corresponding to the diagonal tension assembly under tension to recover to the target length (which is an original length, and can also be other set lengths).

It should be noted that the power source applies a current to the fiber-reinforced composite structure (CFRP fibers), thereby providing cathodic protection to the steel structure column 20. This constitutes a corrosion-resistant cathodic protection system, which can improve the anti-corrosion performance of the steel structure column 20. Additionally, the power source applies an excitation current to the SMA structure (SMA tendon), thereby achieving self-centering of the length of the SMA tendon. This constitutes another self-recovery system with self-centering functionality, which can improve the recoverability of the steel structure column 20 after buckling under external loads (earthquake or impact). Thus, through the cooperation of these two systems, the service life and safety of the steel structure column 20 are improved.

Self-centering of the SMA tendon can be achieved by heating or applying a current to the SMA structure (SMA tendon). In this embodiment, the power source applies an excitation current to the SMA tendon to achieve length self-centering, which can also be understood as regulation through temperature adjustment by applying a current via the power source.

To address the problems of susceptibility to corrosion and lack of self-centering function when existing steel structures buckle in coastal environments or under seismic loads, in the embodiment of the present disclosure spiral grooving is performed (that is, the groove is provided) on the surface of the SMA tendon (namely, the SMA structure) and CFRP is embedded in the groove to form the fiber-reinforced composite structure, thereby realizing an SMA-CFRP tendon (i.e., the diagonal tension assembly) that integrates stress-driven self-centering and cathodic protection functions. The overall technical effects of the anti-buckling system in the embodiment of the present disclosure are as follows: significantly enhancing the anti-buckling capacity of the steel structure, improving the anti-corrosion performance in coastal environments, and increasing the recoverability of the structure after buckling under external loads (earthquake or impact). The anti-buckling system significantly improves the service life and safety of the steel structure, is particularly suitable for coastal and earthquake-prone environments, and reduces the maintenance cost of the structure.

In an embodiment of the present disclosure, as shown in FIG. 1, a first lateral support 21 and a second lateral support 22 are provided on two sides of the steel structure column 20, respectively. The plurality of diagonal tension assemblies includes a first diagonal tension member 11, a second diagonal tension member 12, a third diagonal tension member 13, and a fourth diagonal tension member 14. Two ends of the first diagonal tension member 11 are connected to a top end of the steel structure column 20 and an upper side end of the first lateral support 21 respectively; two ends of the second diagonal tension member 12 are connected to the top end of the steel structure column 20 and an upper side end of the second lateral support 22 respectively; two ends of the third diagonal tension member 13 are connected to a bottom end of the steel structure column 20 and a lower side end of the first lateral support 21 respectively; two ends of the fourth diagonal tension member 14 are connected to the bottom end of the steel structure column 20 and a lower side end of the second lateral support 22 respectively.

It is understandable that the plurality of diagonal tension assemblies in this embodiment includes the four diagonal tension members: the first diagonal tension member 11, the second diagonal tension member 12, the third diagonal tension member 13, and the fourth diagonal tension member 14.

Specifically, in the embodiment of the present disclosure, the SMA-CFRP tendons (diagonal tension assemblies) are installed as an anti-buckling diagonal tension system in the steel structure column 20, and an activation current is applied to generate a pre-tension force.

As shown in FIG. 1, the SMA-CFRP tendon-based anti-buckling diagonal tension system is installed in the steel structure column 20 (which is a rectangular column in this embodiment of the present disclosure). The anti-buckling diagonal tension members of the SMA-CFRP tendons are symmetrically installed on two longer sides of the rectangular column with relatively small moment of inertia, meaning the diagonal tension assemblies are divided into two parts that are installed on two sides of the steel structure column 20, where the two sides are the two long sides with relatively small moment of inertia. Lateral supports made of stainless steel, namely, the first lateral support 21 and the second lateral support 22, are provided in the middle of the rectangular column. The first diagonal tension member 11 (the anti-buckling diagonal tension member of the SMA-CFRP tendon) and the second diagonal tension member 12 are installed on the upper side of the first lateral support 21 and the upper side of the second lateral support 22, respectively. The third diagonal tension member 13 and the fourth diagonal tension member 14 are installed on the lower side of the first lateral support 21 and the lower side of the second lateral support 22, respectively. An angle between the steel structure column 20 (rectangular column) and the first diagonal tension member 11, the second diagonal tension member 12, the third diagonal tension member 13, as well as the fourth diagonal tension member 14 is q (the preset angle in this embodiment is 45 degrees). The first diagonal tension member 11, the second diagonal tension member 12, the third diagonal tension member 13, and the fourth diagonal tension member 14 each have a total length of L. Therefore, the first lateral support 21 and the second lateral support 22 each have a length of sinq*L.

In an embodiment of the present disclosure, the sensing assembly includes a first strain sensor and a second strain sensor. The first strain sensor is disposed on the first diagonal tension member 11, and the second strain sensor is disposed on the second diagonal tension member 12. When the first strain sensor detects that a strain of the first diagonal tension member 11 is greater than a preset value, the power source applies an excitation current to the first diagonal tension member 11 and the third diagonal tension member 13, such that the first diagonal tension member 11 and the third diagonal tension member 13 recover to an initial length. When the second strain sensor detects that a strain of the second diagonal tension member 12 is greater than the preset value, the power source applies an excitation current to the second diagonal tension member 12 and the fourth diagonal tension member 14, such that the second diagonal tension member 12 and the fourth diagonal tension member 14 recover to the initial length. The strain sensor operates by sensing a resistance change in the diagonal tension member.

Specifically, in the self-recovery system with self-centering function of the present disclosure, the strain of the diagonal tension members is monitored by the first strain sensor on the first diagonal tension member 11 and the second strain sensor on the second diagonal tension member 12 to determine their buckling condition. When the strain from the second strain sensor on the second diagonal tension member 12 is less than 0 and the strain from the first strain sensor on the first diagonal tension member 11 is greater than a preset value (which is M_{y}N_{d}l/(12EIEₛA_{b}sinq in this embodiment), where M_{y} is a buckling moment of the rectangular column, N_{d} is a design axial pressure of the rectangular column, l is a height of the rectangular column, EI is stiffness of the rectangular column, Eₛ is an elastic modulus of the SMA-CFRP tendon, A_{b} is an area of the reinforced segment of the SMA-CFRP tendon, and q is an angle between the diagonal tension members and the rectangular column), the rectangular column buckles towards the second lateral support 22. The second diagonal tension member 12 and the fourth diagonal tension member 14 are under compression, while the first diagonal tension member 11 and the third diagonal tension member 13 are under tension. At this time, an activation current can be applied to the first diagonal tension member 11 and the third diagonal tension member 13 under tension to restore them to their original length, realizing the self-centering function of the SMA-CFRP tendon-based anti-buckling diagonal tension system. When the strain of the first diagonal tension member 11 is less than 0 and the strain from the strain sensor of the second diagonal tension member 12 is greater than the preset value (that is, M_{y}N_{d}l/(12EIEₛA_{b}sinq)), the rectangular column buckles towards the first lateral support 21. The first diagonal tension member 11 and the third diagonal tension member 13 are under compression, while the second diagonal tension member 12 and the fourth diagonal tension member 14 are under tension. At this time, an activation current can be applied to the second diagonal tension member 12 and the fourth diagonal tension member 14 under tension to restore them to their original length, realizing the self-centering function of the SMA-CFRP tendon-based anti-buckling diagonal tension system.

In the corrosion-resistant cathodic protection system of the present disclosure, the fiber-reinforced composite structures (that is, CFRP fibers) of the first diagonal tension member 11, the second diagonal tension member 12, the third diagonal tension member 13, and the fourth diagonal tension member 14 are connected to the positive electrode of the power source of the cathodic protection system, and the rectangular column is connected to the negative electrode of the power source of the cathodic protection system. This enables the cathodic protection function of the SMA-CFRP tendon-based anti-buckling system, effectively inhibiting corrosion of the rectangular column.

In an embodiment of the present disclosure, the groove is filled with an insulating layer, and the fiber-reinforced composite structure is insulated from the SMA structure through the insulating layer.

Specifically, a layer of insulating material with a thickness of 0.2 mm, i.e., the insulating layer, is applied between the CFRP fibers and the SMA surface.

In an embodiment of the present disclosure, each of the diagonal tension assemblies includes a reinforced segment, two variable cross-section segments, and two installation segments. The two installation segments are located at two ends; the reinforced segment is located in a middle position; each of the variable cross-section segments connects one end of the reinforced segment and one of the installation segments, and a diameter of the reinforced segment is smaller than a diameter of each of the installation segments.

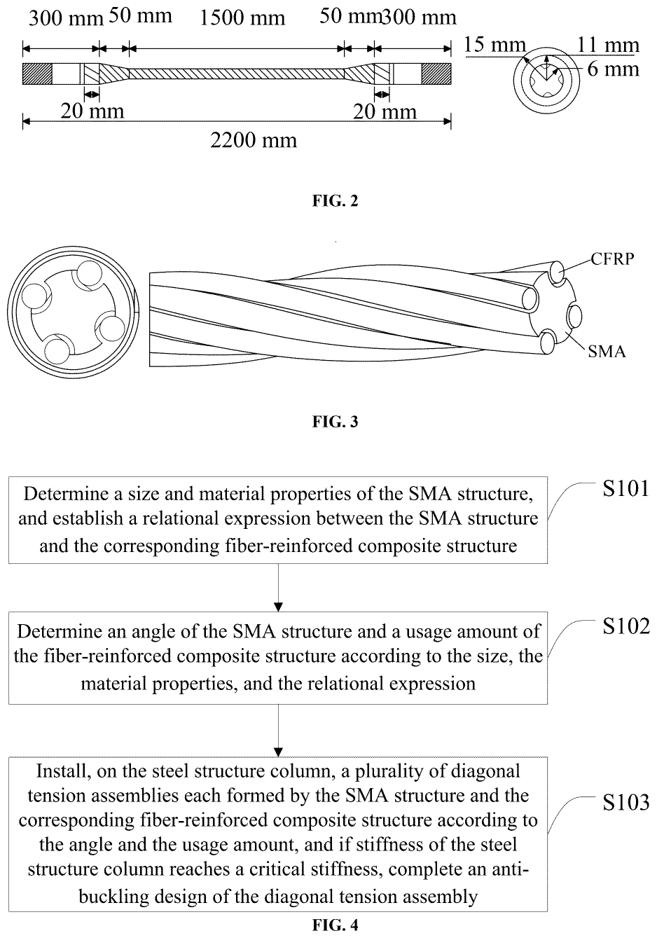

Specifically, as shown in FIG. 2 and FIG. 3, the SMA tendon is divided along a length direction into three parts: the installation segment, the variable cross-section segment, and the reinforced segment. The two ends are the installation segments, each with a length of 300 mm and a diameter of 30 mm. Inward from the two ends are the variable cross-section segments, each with a length of 50 mm. The reinforced segment with a diameter of 12 mm is in the middle, and the length of the reinforced segment can be determined according to actual design requirements (the length is set to 1500 mm in this embodiment of the present disclosure). A diameter ratio of the installation segment to the reinforced segment is 2.5-3. An annular recess with a width of 2 mm and a depth of 4 mm is provided on each of the installation segments, where the annular recess is 20 mm from the variable cross-section segment. Starting from the annular recess, grooves of uniform shape are provided, covering the variable cross-section segment and the reinforced segment. The grooves at the cross-section of the annular recess require chamfering to prevent cutting the CFRP fibers.

In an embodiment of the present disclosure, the groove is a spiral groove, a cross-section of the spiral groove is circular or rectangular, the spiral groove is used for anchoring the fiber-reinforced composite structure, and the fiber-reinforced composite structure has an ultimate strain range of 1.5%-2%.

Specifically, the grooves in the embodiment of the present disclosure are at a 45-degree angle to the horizontal direction, but are not limited thereto. The fiber composite material is CFRP with an ultimate strain of 1.5% to 2% and an elastic modulus of 200 GPa (Gigapascals).

In the embodiment of the present disclosure, the SMA-CFRP tendon is surface-wound with CFRP fibers, which inhibits necking of the SMA tendon under large deformation, thereby significantly improving the strength and ductility of the SMA tendon, making it suitable as an energy-dissipating brace for structural anti-buckling under seismic loads. The CFRP fibers wound on the surface of the SMA-CFRP tendon can serve as auxiliary anodes for cathodic protection, inhibiting structural corrosion and improving the service life of coastal concrete structures. SMA has a stress-driven function, which can generate self-driving stress after structural buckling to enable self-centering of the structure.

Referring to FIG. 2, a preparation method of the SMA-CFRP tendon (diagonal tension assembly) of the present disclosure is as follows:

In step K1, first, to ensure the self-centering capability of the SMA-CFRP tendon, the SMA tendon needs to be pre-tensioned (5% to 6%) before preparation. The SMA tendon selected in the present disclosure is a plain round tendon.

In step K2, a plurality of grooves at a preset angle to the horizontal direction are provided on the surface of the SMA tendon, and the plurality of grooves are filled with fiber composite material. Furthermore, the grooves in the embodiment of the present disclosure are at a 45-degree angle to the horizontal direction, but are not limited thereto. The fiber composite material is CFRP with an ultimate strain of 1.5% to 2% and an elastic modulus of 200 GPa (Gigapascals).

In step K3, the shapes of the provided grooves are the same and can be circular or rectangular; further, the grooves in this embodiment are circular. The grooves need to be arranged at equal intervals both in the SMA cross-section and along the length direction. In this embodiment, four grooves are arranged at equal intervals within the cross-section, and the spacing along the length is 100 mm. The groove width is approximately 2.5 to 3 mm, and the groove volume accounts for approximately 35% to 50% of the volume of the SMA tendon.

In step K4, the SMA tendon is divided along a length direction into three parts: the installation segment, the variable cross-section segment, and the reinforced segment. The two ends are the installation segments, each with a length of 300 mm and a diameter of 30 mm. Inward from the two ends are the variable cross-section segments, each with a length of 50 mm. The reinforced segment with a diameter of 12 mm is in the middle, and the length of the reinforced segment can be determined according to actual design requirements (the length is set to 1500 mm in this embodiment of the present disclosure). A diameter ratio of the installation segment to the reinforced segment is 2.5-3. An annular recess with a width of 2 mm and a depth of 4 mm is provided on each of the installation segments, where the annular recess is 20 mm from the variable cross-section segment. Starting from the annular recess, grooves of uniform shape are provided, covering each variable cross-section segment and the reinforced segment. The grooves at the cross-section of the annular recess require chamfering to prevent cutting the CFRP fibers.

In step K5, the function of the spiral grooves is to anchor the CFRP. CFRP filling starts from the annular recesses at both ends, and to ensure the anchoring effect, CFRP fibers need to be wound at least 2 turns in the annular recesses at both ends.

In step K6, before filling the CFRP fibers, a uniform 0.2 mm thick layer of insulating material needs to be applied on the surface of the SMA tendon.

In step K7, each bundle of CFRP fibers with an area of approximately 2 mm² and a length of 2200 mm is impregnated with epoxy resin, and winding is started from the annular recess at one end of the SMA tendon. First, each bundle of CFRP fibers is wound 2 turns in the annular recess, and then wound along the spiral grooves provided on the variable cross-section segment and the reinforced segment of the SMA tendon to the annular recess at the installation segment of the other end. After 2 turns of winding in the annular recess at the other end, excess CFRP fibers are cut off.

In step K8, the SMA-CFRP tendon wound with CFRP fibers is placed in a curing box at 45 degrees Celsius for 24 hours. The preset angle, the CFRP usage amount, and the stiffness of the obtained SMA-CFRP tendon (diagonal tension assembly) need to satisfy the aforementioned relational expression to prevent necking.

Based on the foregoing embodiments, the present disclosure further provides an anti-buckling design method of the anti-buckling system integrating stress-driven self-centering and cathodic protection functions described above. As shown in FIG. 4, the anti-buckling design method includes the following steps:

In step S101, the size and material properties of the SMA structure are determined, and a relational expression between the SMA structure and the corresponding fiber-reinforced composite structure is established.

In a possible implementation, the material properties include an axial strain at a necking section of the SMA structure, a corresponding cross-sectional pressure at the necking section, and a hardening modulus. The size of the SMA structure is determined based on parameters of the steel structure column; and the axial strain at the necking section of the SMA structure, the corresponding cross-sectional pressure at the necking section, and the hardening modulus are determined based on a stress-strain curve of the SMA structure.

In a possible implementation, the relational expression between the fiber-reinforced composite structure and the SMA structure is as follows:

$${A}_{f} {E}_{f} \{ cos α + \frac{ρ ( {ε}_{y , r} ) - 5 {tan}^{2} {α ( \frac{{r}_{b}}{{r}_{p}} )}^{2} + 5 {tan}^{2} α ( \frac{{r}_{b}}{{r}_{p}} )}{κ ( {ε}_{y , r} ) γ ( {ε}_{y , r} )} \} \geq [ \frac{{A}_{s}}{1 + {ε}_{y , r}} ( \frac{1}{1 + {ε}_{y , r}} ) {σ}_{s} ( {ε}_{y , r} ) - {E}_{t} ] ;$$

where A_{f} is an area of the fiber-reinforced composite structure; E_{f} is an elastic modulus of the fiber-reinforced composite structure; α is a winding angle of the fiber-reinforced composite structure; ε_{y,r} is a strain at a necking section of the SMA structure; ρ(ε_{y,r}) is an axial strain conversion coefficient corresponding to the strain at the necking section of the SMA structure; r_{b} is a radius of the SMA structure; r is a distance from a center of a cross-section of the SMA structure to a center of a cross-section of the fiber-reinforced composite structure; κ(ε_{y,r}) is an elastic modulus conversion coefficient of the fiber-reinforced composite structure corresponding to the strain at the necking section of the SMA structure; γ(ε_{y,r}) is an area conversion coefficient of the fiber-reinforced composite structure corresponding to the strain at the necking section of the SMA structure; As is a cross-sectional area of the SMA structure; σₛ(ε_{y,r}) is a stress corresponding to the strain at the necking section of the SMA structure; and Eₜ is the hardening modulus of the SMA structure.

In other words, the preset angle, the CFRP usage amount, and the stiffness of the SMA-CFRP tendon (diagonal tension assembly) need to satisfy the aforementioned relational expression to prevent necking.

Specifically, in the aforementioned relational expression, the expressions for ρ(ε_{y,r}), κ(ε_{y,r}), and y (ε_{y,r}) are as follows:

$$ρ ( {ε}_{y , r} ) = \sqrt{{ε}_{y , r} + 1} [ ( 2 {tan}^{2} {α ( \frac{{r}_{b}}{{r}_{p}} )}^{2} - 4 {tan}^{2} α ( \frac{{r}_{b}}{{r}_{p}} ) + 2 {tan}^{2} α ) {ε}_{y , r} + 5 {tan}^{2} {α ( \frac{{r}_{b}}{{r}_{p}} )}^{2} - 4 {tan}^{2} α ( \frac{{r}_{b}}{{r}_{p}} ) + 2 {tan}^{2} α ] + [ 5 {tan}^{2} α ( \frac{{r}_{b}}{{r}_{p}} ) ( 1 - ( \frac{{r}_{b}}{{r}_{p}} ) ) ] ;$$

$$κ ( {ε}_{y , r} ) = \sqrt{\begin{matrix}[ 2 {tan}^{2} α ( \frac{{r}_{b}}{{r}_{p}} ) ( 1 - ( \frac{{r}_{b}}{{r}_{p}} ) ) ] \sqrt{{ε}_{y , r} + 1} + {ε}_{y , r}^{3} + 3 {ε}_{y , r}^{2} \\ [ {tan}^{2} {α ( ( \frac{{r}_{b}}{{r}_{p}} ) - 1 )}^{2} + 3 ] {ε}_{y , r} + 2 {tan}^{2} {α ( \frac{{r}_{b}}{{r}_{p}} )}^{2} + 1 ; \\ 2 {tan}^{2} α ( \frac{{r}_{b}}{{r}_{p}} ) + {tan}^{2} α\end{matrix}}$$

$$γ ( {ε}_{y , r} ) = [ 4 {tan}^{2} α ( \frac{{r}_{b}}{{r}_{p}} ) ( ( \frac{{r}_{b}}{{r}_{p}} ) - 1 ) ] \sqrt{{ε}_{y , r} + 1} - 2 {ε}_{y , r}^{3} - 6 {ε}_{y , r}^{2} + [ - 2 {tan}^{2} α ( {( \frac{{r}_{b}}{{r}_{p}} )}^{2} - 2 ( \frac{{r}_{b}}{{r}_{p}} ) + 1 ) - 6 ] {ε}_{y , r} - 2 {tan}^{2} α ( 2 {( \frac{{r}_{b}}{{r}_{p}} )}^{2} - 2 ( \frac{{r}_{b}}{{r}_{p}} ) + 1 ) - 2$$

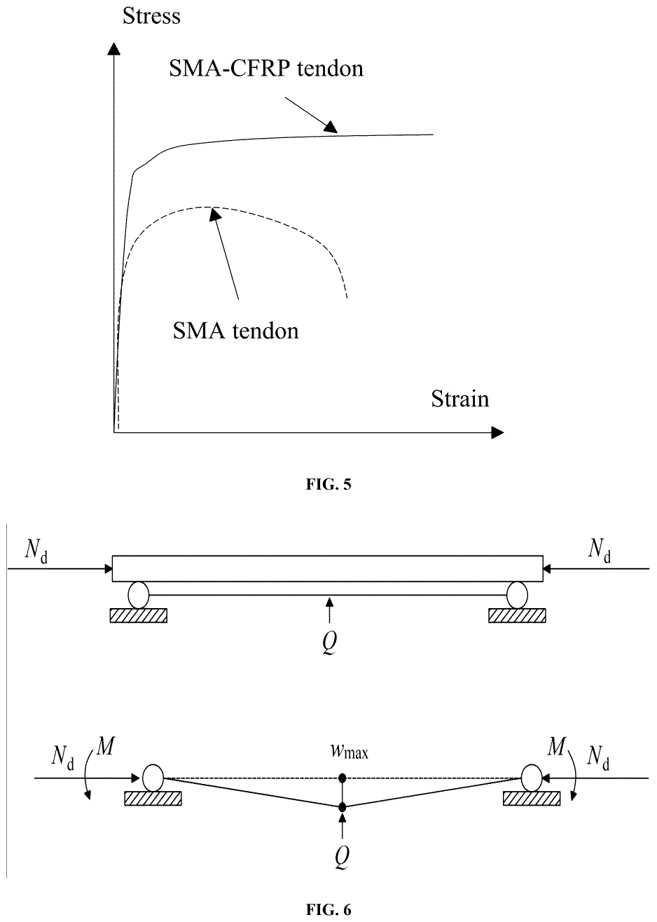

It should be noted that, referring to FIG. 1 and FIG. 5, when the SMA-CFRP tendon (diagonal tension assembly) is in equilibrium or an axial force is less than a pre-tension force of the SMA-CFRP tendon, no deformation occurs. When the load exceeds the pre-tension force of the SMA-CFRP tendon, compressive or tensile deformation appears. Under tension, the SMA-CFRP tendon bears the entire load, dissipating a large amount of energy through its anti-necking, high-strength, and large deformation characteristics, maintaining structural stability. To prevent tensile failure of the SMA-CFRP tendon, the maximum internal stress of the SMA-CFRP tendon should be less than the allowable stress [σ] of the SMA-CFRP tendon.

In the stress-strain curve of the SMA structure in FIG. 5, the arrow position of the SMA tendon corresponds to the necking location. From the curve, the axial strain at the necking section of the SMA structure, the corresponding cross-sectional pressure at the necking section, and the hardening modulus of the SMA tendon are obtained.

That is, in construction of the SMA-CFRP tendon in the present disclosure, the SMA tendon to be reinforced is selected first, and the axial strain at the necking section, the cross-sectional stress corresponding to the strain at the necking section, and the hardening modulus of the stress-strain curve of the necking section are determined based on the local engineering stress-strain curve corresponding to the necking section of the SMA tendon.

In step S102, an angle of the SMA structure and a usage amount of the fiber-reinforced composite structure are determined according to the size, the material properties, and the relational expression.

Specifically, after the geometric size Aₛ of the SMA tendon and the basic material properties ε_{y,r}, σₛ(ε_{y,r}), and Eₜ are determined, an elastic modulus of the CFRP fibers, a fiber winding angle (that is, the preset angle of the grooves on the SMA tendon), and a fiber area can be determined according to the above relational expression. After the winding angle of the CFRP fibers is determined, spiral grooves along the axial direction of the SMA tendon are provided in the variable cross-section segments and the reinforced segment of the SMA tendon.

In step S103, a plurality of diagonal tension assemblies, each formed by the SMA structure and the corresponding fiber-reinforced composite structure according to the angle and the usage amount, are installed on the steel structure column, and if stiffness of the steel structure column (that is, the rectangular column) reaches a critical stiffness, anti-buckling design of one of the diagonal tension assemblies is completed.

In one possible implementation, the critical stiffness is expressed as: K₀=[σ] N_{d}l²/(12 σ_{d}), where K₀ is the critical stiffness, [σ] is an allowable stress of the SMA-CFRP tendon, N_{d} is a design axial pressure of the rectangular column, σ_{d} is a design stress at both ends of the diagonal tension assembly, and l is a height of the rectangular column.

A compressive buckling mechanism based on the SMA-CFRP tendon follows the model shown in FIG. 6. The rectangular column is simply supported, the self-centering anti-buckling force is simplified as a concentrated force, the resultant force of the SMA-CFRP tendons on the tension side acts on the rectangular column through the lateral support, constituting the anti-buckling concentrated force.

When the rectangular column buckles, the stress in the tensioned SMA-CFRP tendon is σ_{d} (design stress). In the critical buckling state, the axial force P in the SMA tendon can be calculated by the following formula:

$$P = {σ}_{max} {A}_{b} ; \qquad ( 1 )$$

where A_{b} is an area of the reinforced segment of the SMA-CFRP tendon, P is the axial force in the SMA tendon, and σₘₐₓ is a stress at both ends.

The anti-buckling concentrated force Q is calculated as follows:

$$Q = 2 {σ}_{max} {A}_{b} sin q ; \qquad ( 2 )$$

where σₘₐₓ is a stress in the tensioned SMA-CFRP tendon, Ap is the area of the reinforced segment of the SMA-CFRP tendon, and q is an angle between the diagonal tension member and the rectangular column.

To prevent buckling, the internal stress σₘₐₓ of the SMA-CFRP tendon calculated in the above formulas (1) and (2) must be less than the allowable stress [σ] of the SMA-CFRP tendon, as expressed in the following formula:

$${σ}_{max} < [ σ ] ; \qquad ( 3 )$$

When σₘₐₓ=[σ], a maximum mid-span deflection wₘₐₓ of the anti-buckling system within an elastic range can be calculated according to the following formula:

$${w}_{max} = \frac{Q {l}^{3}}{48 EI} ; \qquad ( 5 )$$

where EI is stiffness of the rectangular column, and l is a height of the rectangular column.

Substituting formula (2) into formula (5) yields formula (6):

$${w}_{max} = \frac{{l}^{3} [ σ ] {A}_{b} sin q}{24 EI} ; \qquad ( 6 )$$

where EI is the stiffness of the rectangular column, l is the height of the rectangular column, A_{b} is the area of the reinforced segment of the SMA-CFRP tendon, and q is the angle between the diagonal tension member and the rectangular column.

When the SMA-CFRP tendon is in the critical state, the axial stress thereof reaches the design stress σ_{d}. The mid-span displacement is considered to reach its maximum when a plastic hinge forms in the SMA-CFRP tendon. To prevent buckling, a self-regulating moment at the ends should be greater than a bending moment, as shown in the following formula:

$$\frac{{σ}_{d} {A}_{b} sin ql}{2} > {N}_{d} {w}_{max} ; \qquad ( 7 )$$

Substituting formula (6) into formula (7) yields formula (8):

$$EI > \frac{{l}^{2} [ σ ] {N}_{d}}{12 {σ}_{d}} ; \qquad ( 8 )$$

The right side of formula (8) is the critical stiffness. Thus, the critical stiffness of the anti-buckling system can be expressed as: K₀=[σ]N_{d}l²/(12 σ_{d}).

Therefore, when designing the SMA-CFRP tendon-based anti-buckling system, it is only necessary to satisfy the critical stiffness K₀ required to prevent the tensile limit of the SMA-CFRP tendon.

In other words, in the present disclosure, a design equation for the SMA-CFRP tendon is first established based on the SMA-CFRP tendon design equation module, that is, the relational expression. Then, the preset angle and the CFRP usage amount for the SMA-CFRP tendon are determined based on the SMA-CFRP tendon design equation. The prepared SMA-CFRP tendon is used as an anti-buckling diagonal tension member in the structure to construct the anti-buckling system. Next, an anti-buckling equation for the SMA-CFRP tendon under tension is determined, that is, formulas (1) to (8). Finally, the anti-buckling strength of the SMA-CFRP tendon and the stiffness of the rectangular column are determined.

In the present disclosure, through the preparation and design of the SMA-CFRP tendon, the preset angle, the CFRP usage amount, and the stiffness of the SMA-CFRP tendon are calculated according to actual engineering conditions, improving material utilization. The SMA-CFRP tendon of the present disclosure can serve as the diagonal tension member in the structural anti-buckling system. A corresponding design method is established, and a calculation formula for the critical stiffness when the rectangular column in this system buckles is proposed, ensuring the effectiveness of the anti-buckling design.

In the description of the present disclosure, unless otherwise clearly specified, the terms such as “mounting”, “interconnection”, “connection” and “fixation” are intended to be understood in a broad sense. For example, the “connection” may be a fixed connection, a removable connection or an integral connection; may be a mechanical connection, an electrical connection, or mutual communication; may be a direct connection or an indirect connection via a medium; and may be a communication or interaction between two elements. Those of ordinary skill in the art may understand specific meanings of the above terms in the present disclosure based on specific situations.

It should be noted that, in the description of the present disclosure, the terms such as “central”, “longitudinal”, “transverse”, “long”, “wide”, “thick”, “upper”, “lower”, “front”, “back”, “left”, “right”, “vertical”, “horizontal”, “top”, “bottom”, “inner”, “outer”, “clockwise”, “anticlockwise”, “axial”, “radial” and “circumferential” are used to indicate orientations shown in the drawings. It should be noted that these terms are merely intended to facilitate a simple description of the present disclosure, rather than to indicate or imply that the mentioned apparatus or elements must have the specific orientation or be constructed and operated in the specific orientation. Therefore, these terms may not be construed as a limitation to the present disclosure.

In addition, the terms “first” and “second” are merely intended for the purpose of description, and shall not be understood as an indication or implication of relative importance or an implicit indication of a quantity of indicated technical features. Thus, features defined with “first” and “second” may explicitly or implicitly include one or more of the features. In the description of the present disclosure, “a plurality of” means at least two, such as two or three, unless otherwise clearly and specifically limited.

It should be noted that, in the present disclosure, unless otherwise explicitly specified, when it is described that a first feature is “above” or “below” a second feature, it indicates that the first and second features are in direct contact or the first and second features are in indirect contact through an intermediate feature. In addition, when it is described that the first feature is “over”, “above” and “on” the second feature, it indicates that the first feature is directly or obliquely above the second feature, or simply indicates that the altitude of the first feature is higher than that of the second feature. When it is described that the first feature is “under”, “below” or “beneath” the second feature, it indicates that the first feature is directly or obliquely under the second feature or simply indicates that the altitude of the first feature is lower than that of the second feature.

The terms “first”, “second”, “third”, “fourth” and so on (if any) in the specification, claims and the accompanying drawings of the present disclosure are intended to distinguish between similar objects but do not necessarily indicate a specific order or sequence. It should be understood that the data used in such a way may be exchanged under proper conditions to make it possible to implement the described embodiments of the present disclosure in other sequences apart from those illustrated or described here. Moreover, the terms “include”, “contain”, and any other variants mean to cover the non-exclusive inclusion, for example, a process, method, system, product, or device that includes a list of steps or units is not necessarily limited to those steps or units which are clearly listed, but may include other steps or units which are not expressly listed or inherent to such a process, method, system, product, or device.

In the description of this specification, the description with reference to the terms “one embodiment”, “some embodiments”, “an illustrative embodiment”, “an example”, “a specific example”, or “some examples” means that specific features, structures, materials, or characteristics described with reference to the embodiment or example are included in at least one embodiment or example of the present disclosure. In this specification, the schematic representations of the above terms do not necessarily refer to the same embodiment or example. In addition, the specific features, structures, materials, or characteristics described may be combined in any suitable manner in any one or more embodiments or examples.

Finally, it should be noted that the foregoing embodiments are merely used to explain the technical solutions of the present disclosure, but are not intended to limit the present disclosure. Although the present disclosure is described in detail with reference to the foregoing embodiments, the person of ordinary skill in the art should understand that they can still modify the technical solutions described in the foregoing embodiments, or make equivalent substitutions on some or all technical features therein. These modifications or substitutions do not make the essence of the corresponding technical solutions deviate from the spirit and scope of the technical solutions of the embodiments of the present disclosure.

What is claimed is:

1. An anti-buckling system integrating stress-driven self-centering and cathodic protection functions, comprising: a composite tendon, a power source, and a sensing assembly, wherein the composite tendon and the sensing assembly are installed on a steel structure column;
the composite tendon comprises a plurality of diagonal tension assemblies, and the plurality of diagonal tension assemblies are symmetrically distributed on two sides of the steel structure column;
each of the diagonal tension assemblies comprises a shape memory alloy (SMA) structure and a fiber-reinforced composite structure that are insulated from each other; the fiber-reinforced composite structure is disposed in a groove of the SMA structure; two ends of the SMA structure corresponding to each of the diagonal tension assemblies are connected to a positive electrode and a negative electrode of the power source respectively, and the fiber-reinforced composite structure corresponding to each of the diagonal tension assemblies is connected to the positive electrode of the power source; the steel structure column is connected to the negative electrode of the power source; and
when the sensing assembly detects that a strain of the steel structure column reaches a preset condition, the power source applies an excitation current to the SMA structure corresponding to the diagonal tension assembly under tension, such that the diagonal tension assembly under tension recovers to a target length;
a first lateral support and a second lateral support are provided on the two sides of the steel structure column respectively; the plurality of diagonal tension assemblies comprise a first diagonal tension member, a second diagonal tension member, a third diagonal tension member, and a fourth diagonal tension member; two ends of the first diagonal tension member are connected to a top end of the steel structure column and an upper side end of the first lateral support respectively; two ends of the second diagonal tension member are connected to the top end of the steel structure column and an upper side end of the second lateral support respectively; two ends of the third diagonal tension member are connected to a bottom end of the steel structure column and a lower side end of the first lateral support respectively; two ends of the fourth diagonal tension member are connected to the bottom end of the steel structure column and a lower side end of the second lateral support respectively;
the sensing assembly comprises a first strain sensor and a second strain sensor, the first strain sensor is disposed on the first diagonal tension member, and the second strain sensor is disposed on the second diagonal tension member;
when the first strain sensor detects that a strain of the first diagonal tension member is greater than a preset value, the power source applies an excitation current to the first diagonal tension member and the third diagonal tension member, whereby the first diagonal tension member and the third diagonal tension member recover to an initial length; and
when the first strain sensor detects that a strain of the second diagonal tension member is greater than the preset value, the power source applies an excitation current to the second diagonal tension member and the fourth diagonal tension member, whereby the second diagonal tension member and the fourth diagonal tension member recover to the initial length;
the steel structure column is a rectangular column; the first diagonal tension member and the third diagonal tension member are installed on one long side of the rectangular column with relatively small moment of inertia; the second diagonal tension member and the fourth diagonal tension member are installed on another long side of the rectangular column with relatively small moment of inertia;
when the strain from the second strain sensor on the second diagonal tension member is less than 0 and the strain from the first strain sensor on the first diagonal tension member is greater than a preset value, the rectangular column buckles towards the second lateral support; the second diagonal tension member and the fourth diagonal tension member are under compression, while the first diagonal tension member and the third diagonal tension member are under tension; and in this case, an activation current is applied to the first diagonal tension member and the third diagonal tension member under tension to restore the first diagonal tension member and the third diagonal tension member to an original length; when the strain of the first diagonal tension member is less than 0 and the strain from the strain sensor of the second diagonal tension member is greater than the preset value, the rectangular column buckles towards the first lateral support; the first diagonal tension member and the third diagonal tension member are under compression, while the second diagonal tension member and the fourth diagonal tension member are under tension; and then, an activation current is applied to the second diagonal tension member and the fourth diagonal tension member under tension to restore the second diagonal tension member and the fourth diagonal tension member to the original length, wherein the preset value is M_{y}N_{d}l/(12EIEₛA_{b}sinq), M_{y} is a buckling moment of the rectangular column, N_{d} is a design axial pressure of the rectangular column, l is a height of the rectangular column, EI is stiffness of the rectangular column, Eₛ is an elastic modulus of an SMA-carbon fiber reinforced polymer (CFRP) tendon, A_{b} is an area of a reinforced segment of the SMA-CFRP tendon, and q is an angle between the diagonal tension members and the rectangular column; and
the groove is a spiral groove, a cross-section of the spiral groove is circular or rectangular, the spiral groove is used for anchoring the fiber-reinforced composite structure, and the fiber-reinforced composite structure has an ultimate strain range of 1.5%-2%.

2. The anti-buckling system integrating stress-driven self-centering and cathodic protection functions according to claim 1, wherein the groove is filled with an insulating layer, and the fiber-reinforced composite structure is insulated from the SMA structure through the insulating layer.

3. The anti-buckling system integrating stress-driven self-centering and cathodic protection functions according to claim 1, wherein each of the diagonal tension assemblies comprises a reinforced segment, two variable cross-section segments, and two installation segments; the two installation segments are located at two ends; the reinforced segment is located in a middle position; each of the variable cross-section segments connects one end of the reinforced segment and one of the installation segments, and a diameter of the reinforced segment is smaller than a diameter of each of the installation segments.

4. An anti-buckling design method of the anti-buckling system integrating stress-driven self-centering and cathodic protection functions according to claim 1, wherein the anti-buckling design method comprises:
determining a size and material properties of the SMA structure, and establishing a relational expression between the SMA structure and the corresponding fiber-reinforced composite structure;
determining an angle of the SMA structure and a usage amount of the fiber-reinforced composite structure according to the size, the material properties, and the relational expression; and
installing, on the steel structure column, a plurality of diagonal tension assemblies each formed by the SMA structure and the corresponding fiber-reinforced composite structure according to the angle and the usage amount, and if stiffness of the steel structure column reaches a critical stiffness, completing anti-buckling design of one of the diagonal tension assemblies.

5. The anti-buckling design method of the anti-buckling system according to claim 4, wherein the material properties comprise an axial strain at a necking section of the SMA structure, a corresponding cross-sectional pressure at the necking section, and a hardening modulus;
the determining the size and the material properties of the SMA structure comprises:
determining the size of the SMA structure based on parameters of the steel structure column; and
determining the axial strain at the necking section of the SMA structure, the corresponding cross-sectional pressure at the necking section, and the hardening modulus based on a stress-strain curve of the SMA structure.

6. The anti-buckling design method of the anti-buckling system according to claim 4, wherein the relational expression is as follows:
$${A}_{f} {E}_{f} \{ cos α + \frac{ρ ( {ε}_{y , r} ) - 5 {tan}^{2} {α ( \frac{{r}_{b}}{{r}_{p}} )}^{2} + 5 {tan}^{2} α ( \frac{{r}_{b}}{{r}_{p}} )}{κ ( {ε}_{y , r} ) γ ( {ε}_{y , r} )} \} \geq [ \frac{{A}_{s}}{1 + {ε}_{y , r}} ( \frac{1}{1 + {ε}_{y , r}} ) {σ}_{s} ( {ε}_{y , r} ) - {E}_{t} ] ;$$
wherein A_{f} is an area of the fiber-reinforced composite structure; E_{f} is an elastic modulus of the fiber-reinforced composite structure; α is a winding angle of the fiber-reinforced composite structure; ε_{y,r} is a strain at a necking section of the SMA structure; ρ(ε_{y,r}) is an axial strain conversion coefficient corresponding to the strain at the necking section of the SMA structure; r_{b} is a radius of the SMA structure; rₚ is a distance from a center of a cross-section of the SMA structure to a center of a cross-section of the fiber-reinforced composite structure; κ(ε_{y,r}) is an elastic modulus conversion coefficient of the fiber-reinforced composite structure corresponding to the strain at the necking section of the SMA structure; γ(ε_{y,r}) is an area conversion coefficient of the fiber-reinforced composite structure corresponding to the strain at the necking section of the SMA structure; As is a cross-sectional area of the SMA structure; σₛ(ε_{y,r}) is a stress corresponding to the strain at the necking section of the SMA structure; and Eₜ is the hardening modulus of the SMA structure.

7. The anti-buckling design method of the anti-buckling system according to claim 4, wherein the critical stiffness is expressed as:
$${K}_{0} = [ σ ] {N}_{d} {l}^{2} / ( 12 {σ}_{d} ) ;$$
wherein K₀ is the critical stiffness; [σ] is an allowable stress of the diagonal tension assembly; σ_{d} is a design stress at two ends of the diagonal tension assembly; l is a height of a rectangular column; and N_{d} is a design axial pressure of the rectangular column.

8. The anti-buckling design method of the anti-buckling system according to claim 4, wherein the groove is filled with an insulating layer, and the fiber-reinforced composite structure is insulated from the SMA structure through the insulating layer.

9. The anti-buckling design method of the anti-buckling system according to claim 4, wherein each of the diagonal tension assemblies comprises a reinforced segment, two variable cross-section segments, and two installation segments; the two installation segments are located at two ends; the reinforced segment is located in a middle position; each of the variable cross-section segments connects one end of the reinforced segment and one of the installation segments, and a diameter of the reinforced segment is smaller than a diameter of each of the installation segments.
